(19) United States
(12) Patent Application Publication
(10) Pub. No.: US 2008/0312654 A1
(43) Pub. Date: Dec. 18, 2008

(54) Method for Marking Orthopedic Implant

(75) Inventors: Lucas R. Weatherdon, Marquette, MI (US); Gregory D. Bussone, Marquette, MI (US)

Correspondence Address: FITCH EVEN TABIN AND FLANNERY, 120 SOUTH LA SALLE STREET, SUITE 1600, CHICAGO, IL, 60603-3406, US

(73) Assignee: PIONEER SURGICAL TECHNOLOGY, INC., Marquette, MI (US)

(21) Appl. No.: 12/140,097

(22) Filed: Jun. 16, 2008

Related U.S. Application Data
(60) Provisional application No. 60/944,363, filed on Jun. 15, 2007.

(51) Int. Cl. A61B 17/56; A61B 17/00

(52) U.S. Cl. 606/53; 128/898

(57) ABSTRACT

A novel process for marking implants and instruments is provided by masking a portion of the implant or instrument and then applying a treatment to alter the appearance of the unmasked area. The process may be used to mark surfaces not suitable for laser or chemical etching.

### FIELD OF THE INVENTION

The invention relates to surgical implants and tools with surface indicia, as well as methods for marking surgical implants and tools with surface indicia.

### BACKGROUND OF THE INVENTION

Surgical devices, such as implants and instruments, are often marked for a number of reasons. Markings may indicate, for instance, orientation of the instrument or implant, part number, size, or source. For instance, implants with interlocking components are often marked with lines or arrows to indicate the relative orientation of the components to each other or to related insertion instruments. Markings may also indicate the orientation, bending, or deformation of a single component. For example, substantially straight spinal rods may be marked with an axial line from end to end in order to indicate whether or not the rod is deformed (e.g., twisted or bent). Although it is normally difficult to determine upon visual inspection if a spinal rod is deformed, since the rod may twist while continuing to extend essentially straight in the axial direction, an axial line applied to the surface of the rod will provide a reference point from which bending or twisting of the rod's surface area may be determined. In this instance, twisting of the rod without any deflection or deviation from extending axially straight will still cause the line to adopt a generally helical configuration.

Traditionally, markings have been applied by etching into the surface of a surgical implant or instrument. Most metal surgical implants today are marked by laser, wherein a marking (such as a shape, design, or letter) is physically cut into the surface of the implant by using a laser. Chemical etching is also used to engrave markings into the surface of surgical implants. Mechanical etching of implants is also still used to some extent, although this form of engraving has been largely replaced by laser etching, which is far more accurate and precise than traditional mechanical etching.

These traditional forms of marking, however, are inadequate for certain materials and applications. For instance, it is often difficult to visually detect laser markings (markings made by engraving and/or oxidizing a surface with use of a laser) on components made of metals with high luster because of the high reflectivity of the lustrous surface and the reflectivity of the marked region. For example, cobalt-chromium surgical implants, while generally stronger than titanium or stainless steel implants, have a high luster that makes laser markings on the surface of the implant difficult to detect visually. In addition, cobalt-chromium and certain other metals are not receptive to chemical etching. Therefore, traditional forms of marking are not suitable for use on high luster metal components, and particularly cobalt-chromium components.

Additionally, traditional forms of etching all involve cutting or corroding the surface of an implant, which physically weakens the implant. This makes it more difficult, and sometimes impractical, to mark certain implants with traditional laser marking, chemical etching, or mechanical etching. For instance, implant components that are subject to high stress may fatigue more easily and/or fail more quickly when laser marked, requiring design changes such as an increase in the size of the component. In addition, certain types of markings may be impractical due to fatigue concerns. For instance, it is not ordinarily advisable to etch a circumferential marking on a relatively narrow or elongate part, such as a spinal rod, since such an etching leads to a weakening of shear resistance, which may lead to shear failure of the part when under stress.

There remains a need for surgical implants and instruments that are marked and methods of making such marked implants and instruments so that their markings are more visually discernable than implants that use lasers for generating markings, especially on implants of high luster materials. There further remains a need for implants and instruments that are marked in a manner that does not cause mechanical weakening or fatigue of the implant or instrument and methods of making such implants.

### SUMMARY OF THE INVENTION

The present invention is related to methods of marking surgical devices (e.g. implants and instruments) without traditional laser marking or etching into the surface of the device, as well as implants and instruments marked in this manner. In one form of the invention, a masking agent is applied to a metallic surface of a metallic implant at a predetermined area thereon, thus providing a distinct covered surface area as well as an exposed surface area of the remainder of the device, for example. The masking agent may be, for instance, an adhesive compound applied to the metallic surface of the implant or a stencil placed in front of or into contact with the surface of the implant. A treatment, such as a surface dulling or a chemical, surface coloring treatment, is then applied to the surface of the implant, altering the appearance of the exposed surface area while leaving the covered surface area unaffected. The masking agent is then removed, leaving a distinct marking formed by the contrast between the visual appearance of the treated and untreated portions of the surface.

The surface dulling treatment can be achieved by a blasting process that generates a smoother surface where the blasting agent, e.g. aluminum oxide, hits the metallic surface of the surgical device rather than generally deep cuts or gouges thereon as with the prior etching process. Similarly, chemical surface coloring treatment can be achieved by an anodization process that generates a smooth oxide layer from the native metallic surface so that surface material is not removed by cutting into the metallic surface as with the prior etching process.

The actual discrete marking may represent either the treated or untreated surface. In other words, the masking agent may be applied in the form of a discrete indicium (symbol, letter, shape, line, or the like) so that after the treatment the untreated surface is in the form of the discrete indicium, or alternatively the agent may be applied to the majority of the device, leaving a discrete unmasked portion in the form of an indicium.

By eliminating or minimizing etching into the surface of a surgical implant or instrument, visual indicia such as a visually detectable line, symbol, letter, shape, or combination thereof, may be added to implants and instruments without increasing stress points, providing implants and instruments capable of withstanding greater stresses without failure when compared to traditionally etched components. Thus, the present invention is especially desirable when used for portions of implants or instruments that will be subjected to high levels of stress or that are prone to fatigue due to size, shape, or other variables.

The surfaces of a variety of surgical devices, including orthopedic implants and related insertion instruments, may be marked as described above. The masking agent may be any material or device that temporarily covers a portion of the surface area of a surgical instrument or implant. The masking agent may be, for instance, a tape, resin, or other compound releasably secured to the implant or instrument and then mechanically and/or chemically removed after treatment. Alternatively, the masking agent may be a reusable stencil or the like that is placed over the implant or instrument.

The treatment applied may be any treatment that causes a change in the visual characteristics of the surface of the instrument or implant without creating stress risers in the instrument or implant material. For instance, the treatment may be a dulling treatment such as an abrasive blast process (e.g. aluminum oxide abrasive blast followed by glass bead blast finishing) or an anodizing process that provides a visual contrast between the treated portions and the masked untreated portions of the surface area to which the treatment is applied. Although anodizing previously has been used on surgical components, the process has been primarily used for strengthening certain surfaces or color coding the entire external surface of components, and has not been used to provide discrete markings such as lines, arrows, letters, or other indicia that act as substitutes for laser, mechanical, or chemical etchings.

In one form, a masking agent is applied to a predetermined area or portion of the external surface of a lustrous surgical device (e.g. implant or instrument) in order to selectively mask a relatively small portion of the surface area corresponding in configuration to the indicia desired thereon. After application of the masking agent, an abrasive blast is applied to the device in order to dull the finish of the unmasked portion of the device. The masking agent is then removed, revealing a lustrous or shiny marking on the otherwise dulled surface of the device. This lustrous area is readily detected by the naked eye in an operating room environment.

Alternatively, a relatively small area may be left unmarked and then blasted in order to provide a low luster indicium on a high luster device. For instance, a stencil must be placed over the device with one or more openings in the stencil representing a desired indicium. An abrasive blast is then applied to dull the finish of the unmasked portion of the device. After removal of the stencil, a relatively small area of the device is dulled, providing an indicium that is contrasted to the lustrous finish of the rest of the device.

In another form, a masking agent is applied to a surface of a surgical device, such as a relatively matte or low luster metallic surface of a surgical device, so that a predetermined exposed area or portion having the configuration of a desired discrete marking is created. The device is then subjected to color anodization, wherein the device is placed in an electrolytic solution and subjected to an electrical current, with the device acting as the anode of an electrical circuit, so that the exposed surface area of the implant develops a metallic color finish. Upon removal of the masking agent, the previously covered surface area of the implant is the original color of the implant, such as silver or gray, with a discrete metallic color marking or indicium thereon. This process may be used to mark dull or low luster implants or instruments where blast finishing would not provide sufficient contrast.

Alternatively, a small portion of a device may be masked followed by color anodization of the remaining, unmasked surface. In addition, two or more anodization steps may be used to impart two or more different colors on the surface of the device. Portions of the colored surface may also be altered using abrasive blast techniques.

Other treatments that alter the surface appearance of metals and other compounds used as surgical components may also be used in combination with selective masking in order to mark the surface area of surgical implants and instruments.

In another aspect, implants are provided with distinct indicia thereon without generating significant weakness in the body of the implant such as by stress risers and the like that otherwise would reduce the fatigue strength thereof. In a preferred form, the implant is a spinal rod and the indicium is an axial line generated by treating the remainder of the rod surface after masking or otherwise protecting the area at which the line is to be located. In another form, the axial line is generated by treating only the area on the implant body at which the line is to be located. Manifestly, other high strength implants, instruments, and other devices with differently configured indicia are also contemplated. Insertion tools with indicia can be provided in much the same manner as implants.

### BRIEF DESCRIPTION OF THE DRAWINGS

FIG. 1 shows the steps of a general marking process as disclosed herein.

FIGS. 2 a-d show one form of blast finishing process used to provide a marking in the configuration of a masked portion of a rod.

FIGS. 3 a-d show an alternative form of blast finishing process used to provide a marking in the configuration of an unmasked portion of a rod.

FIGS. 4 a-d show an example of an anodization process used to provide a marking in the configuration of an unmasked portion of a rod.

FIGS. 5 a-e show another, two-step anodization process used to provide a marking in the configuration of an unmasked portion of a rod.

### DETAILED DESCRIPTION OF THE PREFERRED EMBODIMENTS

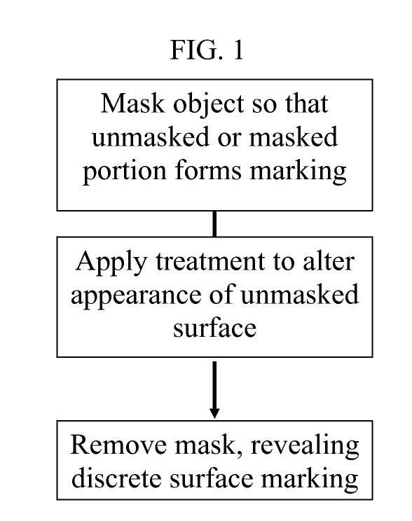

A novel process for marking implants and instruments is provided which includes masking a portion of the implant or instrument and then applying a treatment to alter the appearance of the unmasked area. Masking, as used herein, refers to a process of selectively applying a covering agent to a predetermined portion of a surface, referred to as the masked area, in order to prevent application of a selected treatment to the masked area, as shown generally in FIG. 1. The masking agent protects the masked area from a physical or chemical treatment, so that when the treatment is applied to a surgical implement, only the unmasked or uncovered portion is affected. The masking agent may be, for instance, a removably adhesive compound or a stencil (i.e., a cover with openings forming a specified configuration).

The masking agent is selectively applied so that either the masked portion or unmasked portion creates distinct markings or indicia that convey information to a surgeon using the implant or instrument. For instance, the masking agent may be used to form lines or arrows that indicate orientation of an implant or components of an implant. Markings may be provided to establish orientation with respect to a patient's anatomy, or with respect to other implants, components of an implant, or an insertion tool, such as where markings are provided on two portions of an implant to indicate proper alignment or locking. A marking on an implant may also be configured to identify when the implant is bent, twisted or otherwise deformed with respect to its normal state.

For example, a straight spinal rod will usually need to be bent, twisted and/or curved by a surgeon for being anchored to misaligned vertebral bones. It is desirable for the surgeon to be able to determine if and to what extent the rod has been deformed prior to being locked down in its final implanted configuration. A straight axial line on the rod that a surgeon can see would be desirable in this regard. Any such indicium on the rod should avoid compromising the strength of the rod due to the high loading that is applied thereto both when being deformed and post surgery. To this end the present marked rods have bodies that can withstand such high loads, e.g., while still including axial lines thereon that can be readily identified and seen by the surgeon during a rod implant procedure.

Markings may also be provided on instruments or portions of instruments. For instance, lines may be provided on a tool body and on an actuator so that a surgeon knows when the actuator has been moved a given amount effective for carrying out a desired function.

The masking agent may be applied to the surface to be treated in a number of ways. For instance, the masking agent may be deposited directly onto the surface (for instance, by silk screening or painting one or more layers of masking agent directly to a surface), adhesively secured to the surface (as with removable tapes or resins), or mechanically held to the surface (as in a stencil or physical shield).

After the surface is treated in order to alter the appearance of the unmasked portion, for example, the masking agent may be removed. Some masking agents may be easily removed, such as a stencil that can be reused or other readily removable agent, while others may be removed chemically or through the use of temperature treatments.

One masking agent suited for use with the disclosed process is SpeedMask® UV curable resin (Dymax Corp., Torrington, Conn.). A variety of SpeedMask® resins are available with various properties, and may be quickly applied to metallic surfaces and cured with ultraviolet light to form a protective shell over a preselected portion of a surface. The resins may then be removed by peeling or, in some cases, loosening or dissolving in water. Lacquers, waxes, tapes, and hard fixtures or stencils may also be used to selectively mask portions of a surface.

Traditionally, the masking materials identified above have been used in bonding operations or to cover certain surfaces to provide a coating or finish suitable for a given functional purpose. For instance, in some implants resins have been used to cover interior surfaces of a hollow implant so that only the exterior is anodized. However, masking compounds have not heretofore been used to create discrete markings or indicia on only a portion of a continuous surface, such as the curved, outer surface of a solid spinal rod, to replace laser marking or chemical etching and indicate orientation, size, source, or other similar information.

After masking, a variety of treatments may be used to mark the selectively masked surface. Depending on the material being marked and the nature of the treatment, the marking may comprise distinct indicia formed by the masked portion of the surface, or may comprise negative space constituting unmasked portions of the surface. For instance, in some applications the majority of the surface of the component may be masked, so that only a small portion of the surface area is affected by the treatment, such as when anodization is used to provide color to a discrete, unprotected portion of the surface area or a relatively small portion of a surface area is dulled by blast finishing. In other applications, only a small portion of the surface area is masked, so that discrete indicia design in the shape of the masking agent is formed, such as when a blast treatment is used to dull the majority of the surface of a lustrous surgical implant, leaving a distinct lustrous marking in the shape of the masking agent that contrasts with the relatively matte surfaces provided by blast finishing the remainder of the implant.

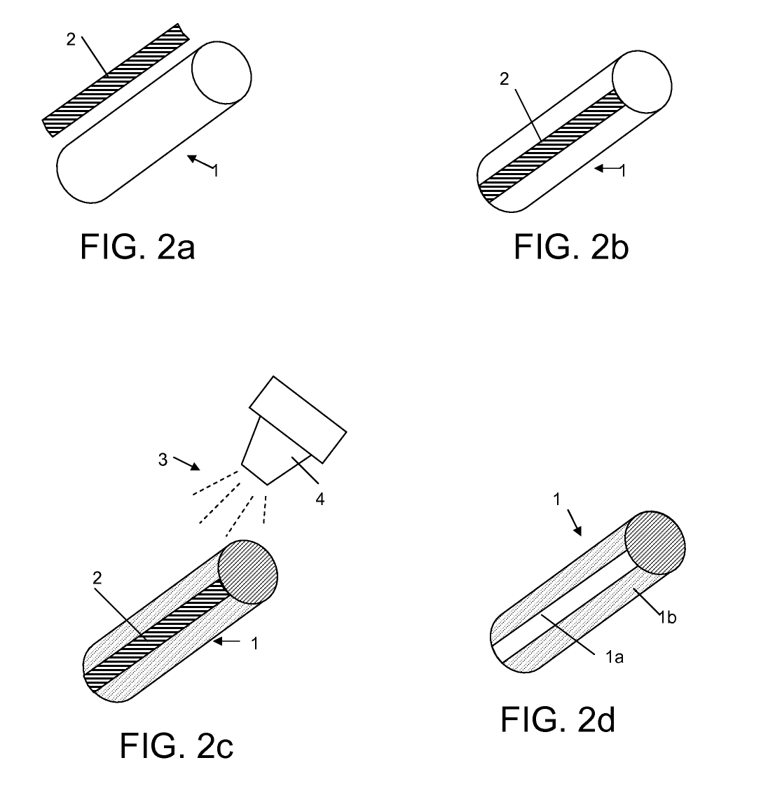

Certain surface treatments are more compatible with certain materials. For instance, high luster surfaces, such as cobalt-chromium, provide glare that makes it difficult to detect when etched mechanically or engraved or oxidized by laser. In addition, cobalt-chromium surfaces are not receptive to chemical treatments such as chemical etching, electroplating, or anodization. However, a blast finishing process may be selectively applied to selectively dull predetermined portions of a cobalt-chromium surface in order to provide a contrast between lustrous and matte portions of the surface, for instance, as shown in the rod marking process shown in FIGS. 2a through 2d.

Other materials may not have the luster of cobalt-chromium, but may be more receptive to chemical treatments such as anodization. For instance, titanium has a relatively matte finish in its natural state, so that abrasive blast finishing will not easily create a visually discernable contrast between treated and untreated areas of the surface. However, titanium is highly receptive to anodization, so that a layer of selected portions may be converted to a color or lustrous appearance.

When using an abrasive blast process, a masking agent is used to shield a portion of a device from treatment. The component should preferably be cleaned prior to application of the masking agent, preferably through a degreasing operation and ultrasonic cleaning, prior to initiation of the blast process. The masking agent may be an adhesive compound releasably adhered to the device. For instance, FIG. 2a shows a cobalt chromium spinal rod 1 and a strip of masking resin 2. The masking resin 2 is shown adhered to the rod 1 in FIG. 2b. The partially masked rod 1 is placed within a blasting apparatus and subjected to a blast of fine particles 3 from a blast nozzle 4 in order to alter the finish of the component, as shown in FIG. 2c. One such fine particle blast procedure is an aluminum oxide blast. Preferably, the blasting agent used is aluminum oxide, due to its low cost, high abrasive potential, and potential for recycling. Aluminum oxide, when used as an abrasive, quickly provides small shallow abrasions on the surface of even hard metals, providing a dull finish while avoiding damage to or weakening of the metal's surface that is often caused by heavier, slower cutting abrasive compounds that cause deeper, concentrated cutting. Thus, a portion of a lustrous metallic surface may be relatively uniformly dulled with aluminum oxide without creating stress risers in the material.

The aluminum oxide blast or other blast procedure may be applied in a tumble blaster, wherein the component to be treated is constantly tumbled so that the aluminum oxide is applied evenly over the entire surface of the component. Alternatively, a cabinet blaster may be used, wherein a stream of aluminum oxide may be directed at a component from a hand-operated nozzle. After the abrasive blast, a blast of glass beads may be used to finish the process and smooth the treated area. In a tumble blaster, the component to be treated is preferably blasted with 80 grit white aluminum oxide at a pressure of 40 PSI for 30 minutes while continuously tumbled, followed by a blast of glass beads at 30 PSI for 10 minutes. In a cabinet blaster process, the component to be treated is preferably blasted with 80 grit white aluminum oxide at a pressure of 40-50 PSI until a uniform surface finish has been applied, followed by blasting with glass beads at a pressure of 40-50 PSI.

After treatment by blast finishing or color anodization to alter the surface appearance of unmasked portions or surface areas of the component, the masking agent may be removed. Some masking agents are readily peeled off of surfaces to which they are applied. Other masking agents are loosened in water or other solvents and after treatment with a solvent may be peeled off of or otherwise mechanically removed from the masked surface. Still other masking agents may be more easily removed when subjected to specific temperatures, and some may even fully dissolve in appropriate solvents.

As shown in FIG. 2d, removal of the masking agent reveals a contrast between the blasted rod surface 1b and the unblasted surface 1a. The fine particle blast provides a matte finish to all non-masked areas of the surface of the rod 1, leaving a discrete, lustrous surface marking 1a in the shape of the applied masking agent once the masking agent is mechanically or chemically removed from the surface of the treated component. Rather than providing a larger, visually detectable cut in the metal surface, resulting in possible stress risers due to the localized removal of relatively significant amounts of material, the blasting process provides numerous small cuts more uniformly across a larger surface area that are individually insignificant, but in the aggregate provide a visually noticeable alteration to the finish of the surface without decreasing the structural integrity of the surface or creating stress risers.

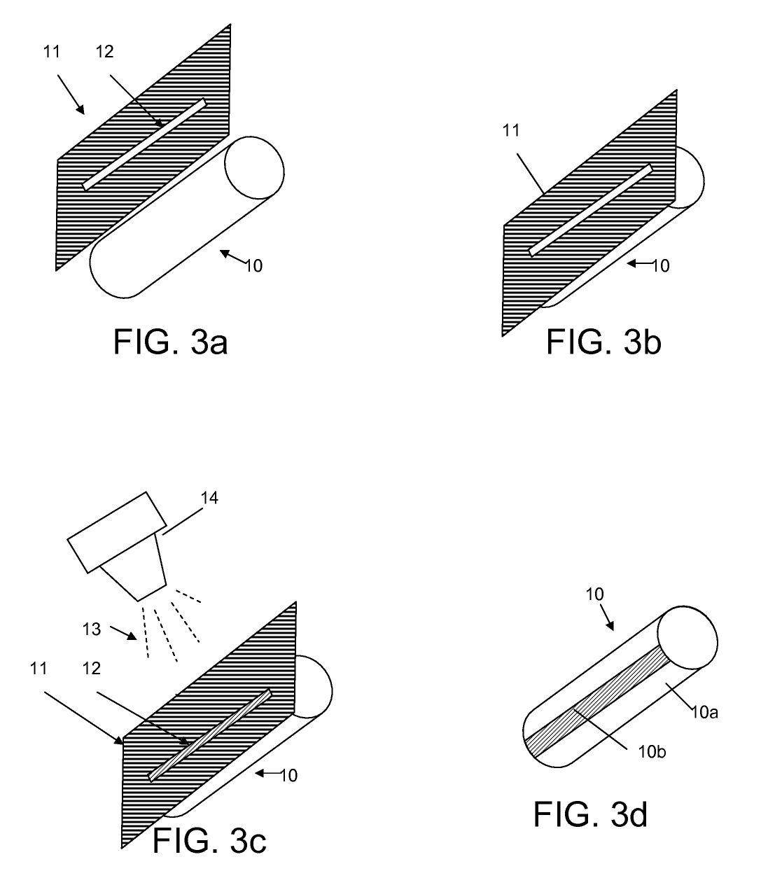

Alternatively, as shown in FIGS. 3a-d, a discrete marking may be formed by the blasted surface area. For instance, a rod 10 may be shielded by a stencil 11, with the stencil having an opening 12 of a desired shape. The stencil (11 is placed in front of the rod 10, as in FIG. 3b, and a particle blast 13 is released from a blast nozzle 14 toward the stencil 11, as in FIG. 3c. After the stencil is withdrawn, the rod surface is marked with an indicium in the shape of the opening in the stencil, providing a visual contrast between the blasted surface of the indicium 10b and the unblasted surface 10a.

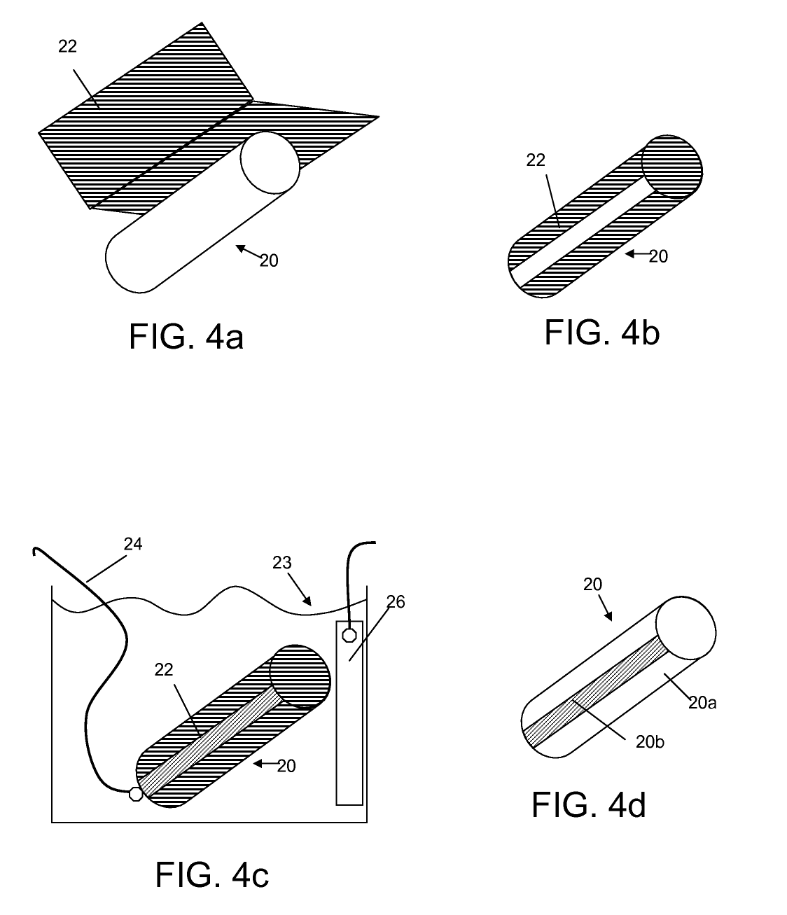

When marking a component with a color anodization procedure, such as the rod marking process shown in FIGS. 4a through 4d, a layer of titanium oxide is formed on the surface of a component electrolytically by using the component to be treated as an anode in an electrical circuit. The color produced by anodization is dependent on the thickness of the titanium oxide layer formed, which may be manipulated by altering the anodizing voltage.

FIG. 4 demonstrates one type of anodization procedure. A device, such as a spinal rod 20, is masked with a masking agent 22, as in FIGS. 4a and 4b. The rod 20 is then placed in an electrolytic solution 23 and connected to a lead 24 from the positive terminal of a source of electrical power, as in FIG. 4c. A cathode 26 in the solution is connected to a negative terminal of the source of electrical power. Passing an electric current through the rod, using the rod as an anode, converts the surface of the unmasked portion of the rod into a colored, oxidized layer. Removing the masking agent reveals a visual contrast between the untreated portion of the rod 20a and the portion with a color anodized layer 20b.

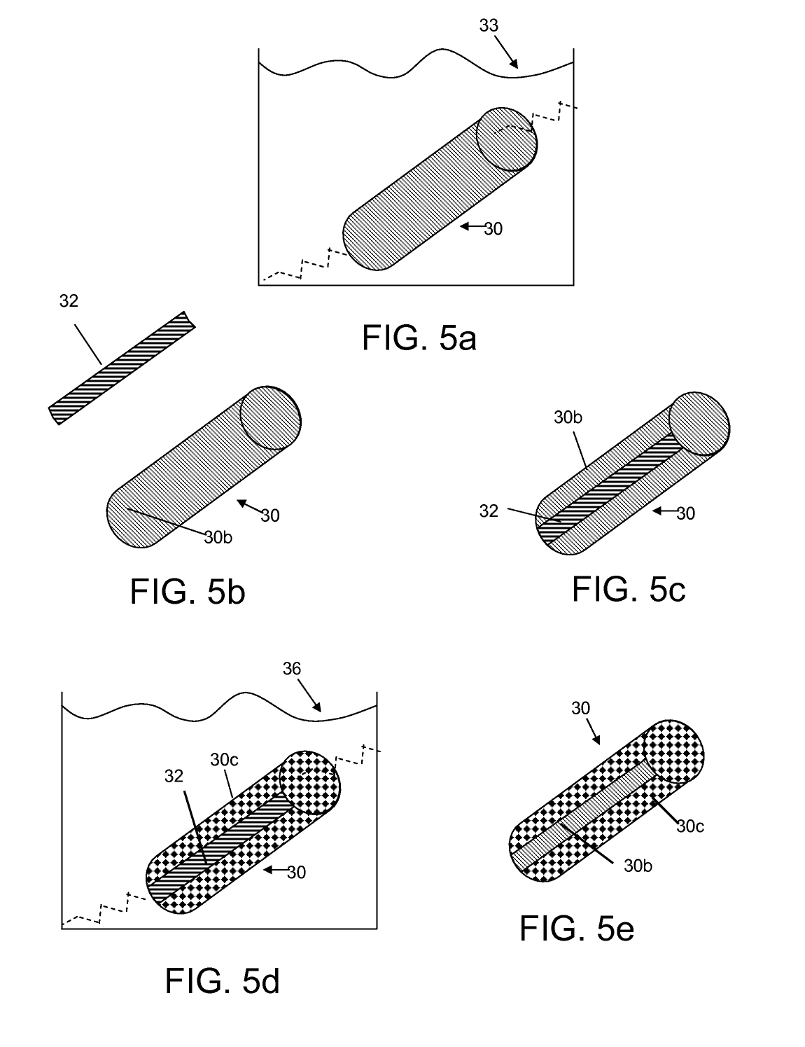

One or more anodization steps may be performed in order to provide multiple colored portions for added visual contrast. FIGS. 5a through 5e demonstrate a two-step anodization procedure. The entire rod 30 is anodized in a first electrolytic solution 33 in FIG. 5a. In FIG. 5b, a masking agent 32 is selected for the anodized rod 30 (having an oxidized layer 30b), and the masking agent is then applied to the rod 30 so that only a portion of the oxidized layer 30b is exposed (FIG. 5c). The rod 30 is then placed in a second electrolytic solution 36 and a current is passed through the rod to form a new oxidized surface 30c on the rod. Removing the masking agent reveals two distinct colored portions 30b and 30c.

The blast finishing and anodization techniques described above may also be combined, for instance by using an abrasive blast to remove portions of an anodized layer to provide color contrast. In one such combination treatment, a device, such as a spinal rod, could be anodized as in FIG. 5a and then selectively masked and subjected to an abrasive blast, such as in FIG. 2c or FIG. 3c, to remove a portion of the colored anodized layer. If an abrasive treatment as in FIG. 2c (where a masking agent is applied in a discrete line along the surface) were used on a colored anodized rod, removal of the masking agent after abrasive blasting would reveal a colored line along a dull spinal rod. Use of the abrasive treatment shown in FIG. 3c (where a stencil having an opening configured as a line is used to shield the rod) would leave an uncolored axial line along the colored anodized rod. This blasting of the anodized article can also provide more accurate, cleaner lines than anodizing unmasked portions of the rod, leaving discrete blasted indicia on an otherwise smooth anodized surface.

Surgical implements marked with the foregoing processes have increased strength compared to laser etched components. In addition, the novel process allows for marking of materials that normally do not produce discernable markings when surface etched with lasers, chemicals, or mechanical means. Using the disclosed methods, markings may be applied to surgical devices where it is impractical using conventional means. For instance, transverse markings may be applied to spinal rods and other narrow elongate surgical implants or devices, even though providing a transverse laser marking might weaken such devices and subjecting them to shearing at relatively low loads of stress.

### Example 1

A 5.75 mm diameter cobalt-chromium spinal rod is marked with a single axial line in order to visually alert a surgeon to torque resulting in twisting of the rod. The rod is ultrasonically cleaned in preparation for masking and aluminum oxide blasting. The rod is placed in a fixture having a slot of a predetermined width. SpeedMask® resin (type 716-R masking agent, Dymax Corp., Torrington, Conn.) is pumped into the slot and into contact with the rod situated within the fixture so that a layer of resin is applied to the rod in the shape and dimensions of the slot. The masking resin applied to the rod is then cured in a UV light chamber equipped with EC5000 bulbs for 99 seconds, and the partially masked rod is removed from the fixture. The partially masked cobalt chromium rod is then placed on a rack and hand-blasted in a blasting cabinet with aluminum oxide at 60 PSI until a uniform matte finish is achieved on the unmasked surface of the rod. The 716-R masking agent is removed by immersing the partially masked rod in hot water and then physically peeling off the masking compound to reveal a lustrous line that contrasts with the matte finish applied to the rest of the rod. The unmasked rod is then ultrasonically cleaned.

A 5.75 mm cobalt-chromium rod marked with the foregoing process has remarkable resistance to fatigue when compared to the same type of rod that is laser etched with a thin axial line. In the comparative laser marking process, a low strength laser was used in order to minimize damage and reduce stress risers to the extent possible, resulting in oxidized laser marking with a minimum of etching or engraving. Under a load of 1775 N, the aluminum oxide blast marked rod held up for at least 5,000,000 cycles, while the laser etched rod showed fatigue after only 77,698 cycles under the same load. Further results are shown in the table below:

| | Cycles for Al—Ox | Cycles for laser- |
|---|---|---|
| | blast marked 5.75 | marked 5.75 mm |
| Load | mm Co—Cr rod | Co—Cr rod |
| 1475 N | N/A | 5,000,000 |
| 1475 N | N/A | 5,000,000 |
| 1575 N | N/A | 98,528 |
| 1775 N | 5,000,000 | 77,698 |
| 1775 N | 5,000,000 | N/A |
| 1975 N | 5,000,000 | N/A |
| 1975 N | 3,574,715 | N/A |
| 2175 N | 1,815,165 | N/A |
| 2275 N | 1,337,980 | N/A |

In addition, the 5.75 mm cobalt-chromium rod with an aluminum oxide blasted line marking shows significantly reduced fatigue when compared to a larger 6.35 mm stainless steel rod. In a four-point bend test, the 5.75 mm cobalt-chromium rod held up for 5 million cycles under a load of 1175 N, while the 6.35 mm stainless steel rod failed at 112,807 cycles under a 1175 N load. Additional results of the four-point bend test comparing the Co—Cr and stainless steel rods are presented below:

| | Cycles for 5.75 mm | |
|---|---|---|
| | Co—Cr rod (Al—Ox | Cycles for 6.35 mm |
| Load | blast marking) | stainless steel rod |
| 1375 N | N/A | 5,000,000 |
| 1475 N | N/A | 5,000,000 |
| 1475 N | N/A | 5,000,000 |
| 1525 N | N/A | 158,139 |
| 1575 N | N/A | 168,350 |
| 1775 N | 5,000,000 | 112,807 |
| 1775 N | 5,000,000 | N/A |
| 1975 N | 5,000,000 | N/A |
| 1975 N | 3,574,715 | N/A |
| 2175 N | 1,815,165 | N/A |
| 2275 N | 1,337,980 | N/A |

As confirmed by these results, the thinner cobalt chromium rod is able to handle more load stress than its stainless steel counterpart. In addition, cobalt-chromium exhibits superior stiffness and reduced spring-back after bending (allowing for more accurate bending). However, adding a laser etched line to the length of the rod significantly decreases fatigue strength of the rod and leads to much more rapid failure under high load stress. By eliminating the laser marking and providing a comparable marking by selectively applying an aluminum oxide grit blast to a partially masked Co—Cr rod, the disadvantages of laser etching may be avoided.

### Example 2

A titanium spinal rod is placed into axial abutment with a fixture having a straight, elongated surface. SpeedMask® resin (type 716-R masking agent, Dymax Corp., Torrington, Conn.) is used to cover the entire surface of the titanium rod except for a long, thin line at the point of abutment between the rod and the fixture. The masking resin applied to the rod is then cured in a UV light chamber equipped with EC5000 bulbs for 99 seconds, and the rod is removed from the fixture. The partially masked titanium rod is then placed on a rack, submerged in a sodium bicarbonate solution, coupled to the anodic terminal of an electrical power source, and anodized at a preselected voltage to create a colored oxidized layer on the unmasked portion of the rod, the color change dependent on the selected voltage. The 716-R masking agent is removed by immersing the partially masked rod in hot water and then physically peeling off the masking compound, resulting in a dull gray titanium rod with a colored line running axially along its surface. The unmasked rod is then ultrasonically cleaned.

While there have been illustrated and described particular embodiments of the present invention, it will be appreciated that numerous changes and modifications will occur to those skilled in the art, and it is intended in the appended claims to cover all those changes and modifications which fall within the true spirit and scope of the present invention.

What is claimed is:

1. A method of marking a surgical device comprising:
selectively shielding one or more portions of a metallic surface of a metal surgical device so that the surface has at least one shielded portion and at least one unshielded portion; and
applying a treatment that alters the physical appearance of the at least one unshielded portion to form at least one discrete indicium on the metallic surface.

2. The method of claim 1, wherein the discrete indicium is a line, symbol, letter, number, or combination thereof.

3. The method of claim 1 wherein the device is of a type intended to be subjected to high stress when in use.

4. The method of claim 1 wherein the device is a cobalt-chromium spinal rod.

5. The method of claim 4 wherein the cobalt-chromium spinal rod is intended to be subjected to stress of at least 1575 Newtons when in use.

6. The method of claim 1 wherein the shielding is accomplished with a resin, tape, or stencil.

7. A method of marking a surgical device, the method comprising:
masking a portion of a surface of the device with a masking agent and leaving an unmasked portion of the surface,
applying an abrasive treatment to the device sufficient to alter the physical appearance of the unmasked portion of the surface;
removing the masking agent to uncover the masked portion of the surface corresponding to a discrete marking on the surface.

8. The method of claim 7 wherein the step of masking is performed by applying a removeable adhesive masking material to selected portions of the surface of the metal surgical device.

9. The method of claim 7 wherein the step of masking is performed by applying a stencil or preformed plate having one or more openings therein to selected portions of the surface of the metal surgical device.

10. The method of claim 7 wherein the surgical device is comprised of titanium and the treatment comprises color anodization.

11. The method of claim 7 wherein the surgical device is comprised of cobalt-chromium and the treatment comprises aluminum oxide blasting.

12. A method of marking a cobalt-chromium spinal rod comprising the steps of:
applying a masking agent to selected portions of a surface of the spinal rod;
applying an electro satin blast to the spinal rod;
removing the masking agent.

13. The method of claim 12 wherein the masking agent is a resin or tape.

14. The method of claim 12 wherein the masking agent is printed onto the surface of the spinal rod.

15. The method of claim 12 wherein the masking agent is applied to form a masked axial line along the length of the rod.

16. The method of claim 12, wherein the masking agent is applied to form a masked transverse or circumferential line on the spinal rod.

17. The method of claim 12, wherein the masking agent is applied in the shape of a letter or number.

18. A surgical device capable of bearing a maximum load, the device having an indicium thereon that does not significantly decrease the maximum load that may be borne by the device.

19. The device of claim 18, wherein the surgical device has a first color and the indicium comprises a second color visually distinct from the first color.

20. The device of claim 19, wherein the surgical device has a first levels of luster and the indicium comprises a second levels of luster visually distinct from the first levels of luster.

21. The device of claim 18, wherein the indicium comprises a discrete line, symbol, letter, number, or combination thereof.
